(19) United States
(12) Patent Application Publication
(10) Pub. No.: US 2008/0243244 A1
(43) Pub. Date: Oct. 2, 2008

(54) Metallic Endoprosthesis Compatible with Magnetic Resonance

(76) Inventors: Arno Bucker, Duren (DE); Alexander Rubben, Aachen (DE)

Correspondence Address: CASELLA & HESPOS, 274 MADISON AVENUE, NEW YORK, NY, 10016, US

(21) Appl. No.: 12/035,699

(22) Filed: Feb. 22, 2008

Related U.S. Application Data
(62) Division of application No. 10/486,023, filed on Mar. 24, 2004, now abandoned.

(30) Foreign Application Priority Data
Aug. 8, 2001 (DE) 201 12 762.8
Dec. 14, 2001 (DE) 201 20 222.0
Feb. 3, 2002 (DE) 202 01 637.4

(51) Int. Cl. A61F 2/06

(52) U.S. Cl. 623/1.53

(57) ABSTRACT

A metallic endoprosthesis is provided and causes no significant artefacts on images taken by magnetic resonance tomography (MRT), as a result of the combination of the production materials with a special design, which permits an evaluation of the externally adjacent region and the lumen of the endoprosthesis by means of MRT. The endoprosthesis is made from a material with a magnetisability similar to human tissue. The design of the endoprosthesis is such that the members or wires of the endoprosthesis run extensively along the longitudinal axis of the endoprosthesis, without forming a closed circuit in a plane which is essentially perpendicular to the endoprosthesis longitudinal axis. Further variations of the endoprosthesis design are possible, which all offer a full compatibility with MR for the endoprosthesis

This application is a divisional of U.S. patent application Ser. No. 10/486,023, filed on Mar. 24, 2004.

### BACKGROUND OF THE INVENTION

1. Field of the Invention

The present invention concerns an endoprosthesis which is MR-compatible.

2. Description of the Related Art

Endoprostheses are used at the present time for example in the region of the vessels (arterial and venous), the bile ducts, the airways and the gastrointestinal tract in order to keep cavities open. The different indications have resulted in the development of various types of endoprostheses in respect of their design configuration and their form. In addition the endoprostheses are produced from the most widely varying materials. Particularly for the intravasal application of endoprostheses—so-called stents—hitherto metal has proven to be the most suitable material. Accordingly, the predominant number of stents are made from metal alloys such as for example high-quality steel or Nitinol. Stents can both be lasered from flat sheets or tubes (U.S. Pat. No. 4,733,665 A) and also woven or braided from wires (U.S. Pat. No. 4,922,905 A). If endoprostheses are produced from non-ferromagnetic metal alloys, then patients can in principle be investigated after the placement of endoprostheses even in strong magnetic fields as there is no fear of movement of the endoprostheses due to positioning in the magnetic field for example of a magnetic resonance (MR) tomograph (Shellock F G, Shellock V J Metallic stents: evaluation of MR imaging safety, AJR Am J Roentgenol 1999; 173:543-7; Hug J, Nagel E, Bornstedt A, Schnackenburg B, Oswald H, Fleck E. Coronary arterial stents: safety and artefacts during MR imaging. Radiology 2000; 216:781-7). That property is nowadays readily referred to as MR-compatibility—more precisely MR-compatibility of the first kind (Schenck J F The role of magnetic susceptibility in magnetic resonance imaging: MRI magnetic compatibility of the first and second kinds. Med Phys 1996; 23: 815-850). In that respect it will be appreciated that only the absence of risk to the patient is taken into consideration if the patient is exposed to a strong magnetic field after implantation of the MR-compatible endoprosthesis.

Previously used endoprostheses of metal produce artefacts in the magnetic resonance tomography image, which, particularly in the case of relatively small vessels, do not permit evaluation of the lumen of an endoprosthesis by means of magnetic resonance tomography (Meyer J M, Buecker A, Schuermann K, Ruebben A, Guenther R W, MR evaluation of stent patency: In vitro tests of 22 metallic stents and the possibility of determining their patency by MR angiography. Invest Radiol 2000; 35:739-746; Klemm R, Duda S, Machann J, et al, MR imaging in the presence of vascular stents: A systematic assessment of artefacts for various stent orientations, sequence types, and field strengths. J Magn Reson Imaging 2000; 12-606-15). That is caused both by the differing magnetisability of the metal alloys used, in comparison with human tissue, and also eddy currents or radio frequency effects (Lüdecke K M, Röschmann P, Tischler R, Susceptibility artefacts in NMR imaging. Magn Reson Imaging 1985; 3:329-343; Camacho C R, Plewes D B, Henkelman R M; Nonsusceptibility artefacts due to metallic objects in MR imaging. J Magn Reson Imaging 1995; 5:75-88). It will be noted that there is also a diagnostic approach which under certain conditions uses the stent actively for MR-imaging (U.S. Pat. No. 6,280,385, EP 1 023 609 B1, WO 99/19738 and Quick H H, Ladd M E, Nanz D, Mikolajczyk K P, Debatin J F. Vascular stents as RF antennas for intravascular MR guidance and imaging. Magn Reson Med 1999; 42:738-45).

Therefore the object of the present invention is to provide endoprostheses which do not suffer from the above-indicated disadvantages such as the creation of artefacts in the magnetic resonance tomography image and endangerment to the patient in investigation by means of magnetic resonance tomography—more specifically as far as possible independently of the MR-technology used. In that respect the invention seeks to provide that it is possible to evaluate both the tissue which is disposed externally around the endoprosthesis and also the lumen of the endoprosthesis, by means of the magnetic resonance tomography images. The invention further seeks to provide that, together with the lack of ferromagnetic properties, those metallic endoprostheses can be identified as ‘fully MR-compatible’, which corresponds to the scientifically described requirement of MR-compatibility of the second kind.

### SUMMARY OF THE INVENTION

This invention involves an endoprosthesis comprising a metallic material which has a magnetic susceptibility in the range of between −300×10⁻⁶ and 300×10⁻⁶, wherein the endoprosthesis is of such a configuration that individual endoprosthesis bars or wires are so oriented along the longitudinal axis of the endoprosthesis that they form substantially no continuous electrical circuit in a plane which is oriented substantially perpendicularly with respect to the longitudinal axis of the prosthesis, over the circumference of the endoprosthesis.

### BRIEF DESCRIPTION OF THE DRAWINGS

The invention is further described with reference to the drawings in which:

FIG. 1:

The drawing shows a three-dimensional model of a possible fully MR-compatible design which can be produced for example from a tube or flat sheet. The endoprosthesis bars 1 which start from a helical backbone 3 do not form closed circuits. The Figure shows mutually opposite eyes 2 for possibly fixing a connection of the endoprosthesis bars 1 to the backbone 3 by a structure which is non-conducting or which is only a very poor conductor.

FIG. 2a:

This Figure shows a possible design (two-dimensional) of a fully MR-compatible endoprosthesis (non-expanded) which can be produced for example from a tube or flat sheet. The endoprosthesis bars 1 which start from a helical backbone 3 do not form closed circuits. The Figure shows mutually opposite eyes 2 for possibly fixing a connection of the endoprosthesis bars 1 to the backbone 3 by a structure which is non-conducting or which is only a very poor conductor.

FIG. 2b:

This Figure shows a possible design (two-dimensional) of a fully MR-compatible endoprosthesis (non-expanded) which can be produced for example from a tube or flat sheet. The endoprosthesis bars 1 which start from a helical backbone 3 do not form closed circuits. The Figure shows mutually opposite eyes 2 for possibly fixing a connection of the endoprosthesis bars 1 to the backbone 3 by a structure which is non-conducting or which is only a very poor conductor.

FIG. 2c:

This Figure shows a possible design (two-dimensional) of a fully MR-compatible endoprosthesis (non-expanded) which can be produced for example from a tube or flat sheet. The endoprosthesis bars 1 which start from a helical backbone 3 on the one hand do not form closed circuits 4 and on the other hand form a closed circuit 5. To maintain full MR-compatibility the closed circuits 5 must in part comprise intermediate portions 6 which are very poor conductors or not electrically conducting (1).

FIG. 2d:

This Figure shows a possible design (two-dimensional) of a fully MR-compatible endoprosthesis (non-expanded) which can be produced for example from a tube or flat sheet. The endoprosthesis bars 1 which start from a helical backbone 3 do not form closed circuits. The endoprosthesis bars 1 form respective polygonally shaped, mutually opposite pairs 7.

FIG. 2e:

This Figure shows a possible design (two-dimensional) of a fully MR-compatible endoprosthesis (non-expanded) which can be produced for example from a tube or flat sheet. The polygonal endoprosthesis bars 1 which start from a helical backbone 3 do not form closed circuits. The endoprosthesis bars 1 are in mutually displaced relationship in a sawtooth-like configuration.

FIGS. 3a and 3b:

A three-dimensional model as a plan view (a) and a profile view (b), showing the connection 8 (dark grey), which is not true to scale, between an endoprosthesis bar 1 and a helical backbone 3. That connection should be as flat as possible and must comprise a material which is either not conducting or which is only a very poor electrical conductor. The required minimum length of the connecting portion 8 which is not electrically conducting or which is only a very poor electrical conductor depends on whether the metallic material is electrically insulated or not.

FIGS. 4a-g:

Three-dimensional diagrammatic drawings of a fully MR-compatible endoprosthesis (not true to scale) which can be produced for example from a tube or flat sheet. The endoprosthesis bars 1 start for example from a single straight backbone 3. The endoprosthesis bars 1 can be displaced in a sawtooth-like configuration and can start straight alternately from respective sides of the backbone 3 (4a, b, c) or however at an angle relative to the line of the backbone 3 (4d). Advantageously the endoprosthesis bars 1 form loops which originate again alternately from one side or the other of the backbone 3 (4e, f). The shape of the endoprosthesis loops is advantageously polygonal (4g).

FIG. 5:

Diagrammatic drawings demonstrating by way of ample possible shapes of the backbone 3 (FIGS. 5a, b) or the backbones 3 (FIGS. 5c, d) of the fully MR-compatible endoprosthesis. Advantageously, the backbone 3 is of a helical shape (5a, b). When a plurality of backbones 3 are used the helical shape can be retained for all backbones 3, in which respect there are as few intersection points of the backbones 3 as possible and they should be as far away from each other as possible (5c, d).

FIG. 6:

Magnetic resonance tomography images of stents which were positioned in a water bath and then measured. The orientation of the stents is perpendicular to the main magnetic field axis in order to provoke the greatest artefacts.

FIG. 6a:

Braided stents of gold with insulation (1), of copper without insulation (2) and of copper with insulation (3). The insulated copper stent is almost invisible (3) and the insulated gold stent does not exhibit any substantial artefacts which go beyond the wall of the endoprosthesis (1). The importance of the insulation will be clear on the basis of the large artefact of the uninsulated copper stent (2) as, by virtue of its susceptibility which is almost identical to human tissue, copper does not cause any susceptibility artefacts.

FIG. 6b:

Woven stents of various diameters comprising a palladium-silver alloy with insulation, which exhibit slight but still acceptable artefacts, at small diameters.

FIG. 6c:

Lasered stents comprising a copper-gold alloy with (14) and without (5) closed electrically conducting structure over the entire circumference of the endoprosthesis. All endoprostheses with the closed conducting structure almost perpendicularly to the longitudinal axes of the endoprostheses exhibit pronounced artefacts in a direct comparison with an endoprosthesis without a closed conducting structure (5).

FIG. 6d:

Transversely braided stents (1-3) and predominantly longitudinally braided stent (4), in each case of copper. All transversely braided stents exhibit marked artefacts (1-3) while the longitudinally braided stent (4) is artefact-free and is almost invisible in the MR-image, which proves the significance of the correct design for MR-compatibility.

FIG. 7:

Magnetic resonance tomography images of pigs after stent placement, which were recorded with a coronary angiography MR sequence.

FIG. 7a:

Two high-quality steel stents placed in the left descending coronary artery (LAD) exhibit pronounced artefacts which permit neither evaluation of the stent lumen nor the surroundings of the coronary in the proximity of the stent.

FIG. 7b:

A braided stent placed immediately behind the clearly visible exit of the sinus node artery in the right coronary artery cannot be seen on the MR-image, which permits artefact-free evaluation both of the stent lumen and also the area around the stent.

FIG. 7c:

A stent which is placed in the proximal region of the LAD and which is woven along the longitudinal axis of the stent cannot be seen on the MR-image, which permits artefact-free evaluation both of the stent lumen and also the area around the stent.

FIG. 7d:

A lasered stent which is placed in the proximal region of the LAD, without closed electrically conducting circuits, cannot be seen on the MR-image, which permits artefact-free evaluation both of the stent lumen and also the area around the stent.

FIG. 8:

Renal arteries after placement of MR-compatible stents in the right and left renal arteries of a pig and representation by means of various MR-angiography (MRA) procedures: spin labelling (a), phase contrast angiography before (b) and after (c) stenting, and contrast agent-enhanced T1 gradient echo sequence (d). The phase contrast angiographies were implemented for direct comparison before and after stenting. On none of the MR-images is it possible to see an artefact which would interfere with the image or which even only permits location of the stent. X-ray angiography after contrast agent administration shows the position of the stents in the renal arteries (e, arrows).

### DETAILED DESCRIPTION OF THE PREFERRED EMBODIMENTS

Imaging in nuclear spin tomography involves using magnetic fields of 0.064 to 3 Teslars and in part also above that value. What is important in this connection is in particular the representation of the arteial and venous vessels as well as the imaging of the bile ducts which have become established in clinical application. If materials of different magnetisability (magnetic susceptibility) are in immediate proximity, so-called susceptibility artefacts occur. They give rise in the MR-image to signal extinction phenomena and distortion effects which make it impossible to effect evaluation in that region of the MR-image.

The inventors realised that, to avoid excessively large artefacts in relation to endoprostheses, they should be made from materials which are of a magnetisability (magnetic susceptibility) which is similar to human tissue. For example copper, gold, copper-gold alloys and palladium-silver alloys were found to be suitable if in addition the prerequisites described hereinbelow for a fully MR-compatible design are observed. It will be noted that, besides susceptibility artefacts, artefacts can nonetheless still occur due to the formation of eddy currents and radio frequency effects, such as for example screening of the interior of an endoprosthesis.

Now, in accordance with the present invention, the inventors realised that the combination of metals or metal alloys without a substantial susceptibility difference in relation to human tissue with the specific designs of an endoprosthesis substantially prevents the occurrence of any artefacts in the MR-image. To prevent the artefact-generating flow of eddy currents or radio frequency shielding, the possibility of a completely circulating flow of current, in particular in a plane which is oriented substantially perpendicularly to the longitudinal axis of the endoprosthesis, should be precluded.

An endoprosthesis according to the invention can be produced by any manner of manufacture known to the man skilled in the art. Suitable manufacturing methods are described in U.S. Pat. No. 4,733,665 A, U.S. Pat. No. 4,922,905 A and Palmaz, Cardiovasc. Intervent. Radiol. 1992, 15:279-284, in which respect those disclosures are incorporated herein by reference. It has proven to be advantageous if, besides braided or woven wires, flat sheets or tubes are lasered to produce endoprostheses. Irrespective of the manner of manufacture (lasering versus braiding/weaving), a material should be used, which generates no or only minimal susceptibility artefacts.

In particular implants with those properties are suitable for use in human or animal vessels, vessel bypasses, ureters, intrahepatic bypasses, bile ducts and for use in other hollow organs.

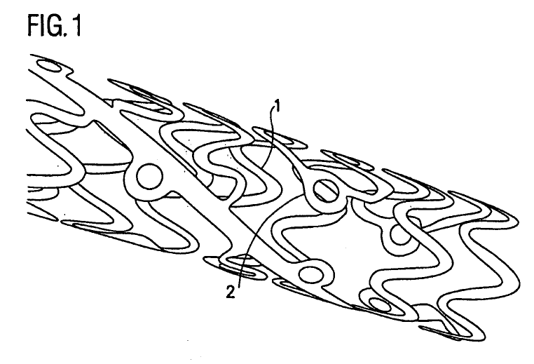

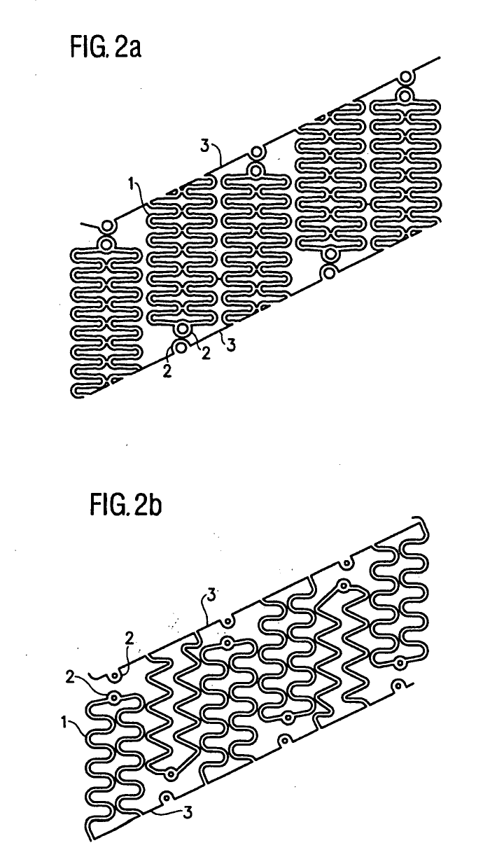

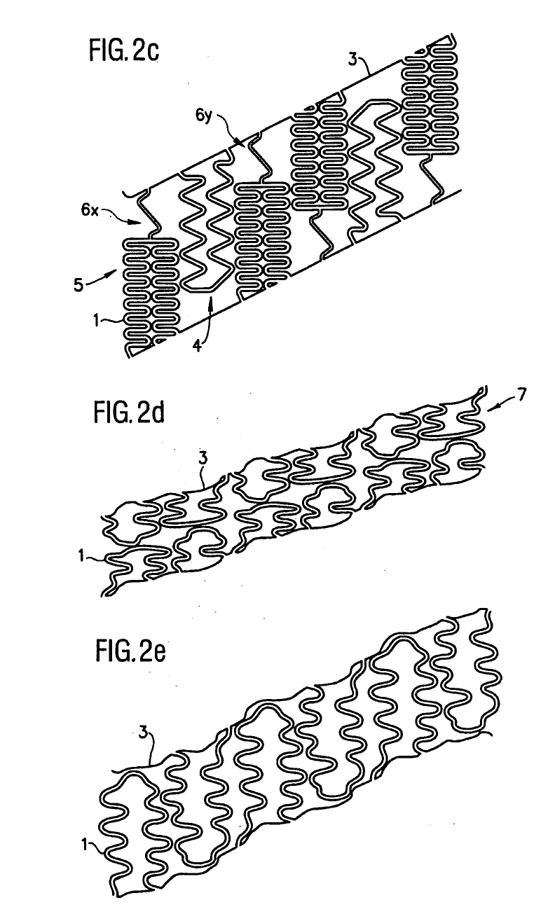

A preferred manner of manufacture for the endoprostheses according to the invention is lasering which is described in greater detail hereinafter in respect of MR-compatibility. Various endoprosthesis designs can be considered in the case of lasering of the endoprostheses. It has proven to be particularly advantageous if the individual endoprosthesis bars extend from one or more backbones, without the bars or the metallic parts of those prosthesis bars being able to form a continuous conducting circuit in a plane substantially perpendicularly to the longitudinal axis of the endoprosthesis over the entire circumference thereof. That arrangement on the one hand prevents local magnetic fields being built up by eddy currents while on the other hand it provides for shielding the interior of the prosthesis from the radio frequency energy which is radiated in the context of MR-imaging. The backbone or backbones can be straight or can be of any shape, in which respect in particular a helix is advantageous (FIGS. 1, 2 and 3). The endoprosthesis bars can be of any shape, which includes individual bar-like or curved struts and also straight or curved (bent) double struts arranged in a semicircular configuration (FIGS. 2a-e, 4a-g). The curvature or bend of individual or double struts can assume any shape in that respect, advantageously having regard to the above-indicated prerequisites. Individual and double bars can also be used in combination. Thus the endoprosthesis does not produce any artefacts worth mentioning in the MR-image, which in particular also permits evaluation of the interior of the endoprosthesis by means of magnetic resonance tomography. In a preferred embodiment disposed between the endoprosthesis bars are connecting bars which are non-conducting or only slightly current-conducting.

A further preferred manner of manufacture is braiding or weaving, which is described in greater detail hereinafter in respect of MR-compatibility. If the endoprosthesis is to be braided or weaved from a wire, then the eddy currents which occur should also be reduced or deflected, to such an extent that no troublesome magnetic fields occur or radio frequency shielding effects arise. For that purpose advantageously on the one hand the wires are so insulated that no conducting connections are present at the points of contact of the wires and on the other hand each individual wire is oriented as much as possible on the longitudinal axis of the endoprosthesis so that in particular no closed or almost closed circuit is formed in a plane substantially perpendicularly to the longitudinal axis of the endoprosthesis and over the entire circumference thereof. That principle is independent of whether only a single wire or a plurality of wires are used to, produce the endoprosthesis. It is equally immaterial whether the arrangement of the wires is achieved by braiding or weaving. In that case, the wire or wires can assume zig-zag, omega, sinusoidal or other polygonal shapes as long as the main orientation is along the longitudinal axis of the endoprosthesis.

The invention involves endoprostheses which can be manufactured from various metallic magnetic resonance-compatible materials. Those materials are metals or metal alloys which are distinguished in that, by virtue of a magnetisability which is similar to human tissue, no substantial susceptibility artefacts are produced in MR-images. Those alloys preferably involve copper-bearing, silver-bearing, palladium-bearing or gold-bearing metal mixtures. In addition the pure substances and in that respect in particular copper are also suitable as the material for making the endoprosthesis. As the extent of possible susceptibility artefacts, besides the difference in magnetisabilities of two substances, is also dependent on further factors and minimal artefacts in the MR-image can be tolerated, it is not possible to specify absolute fixed limit values. Magnetic susceptibility in accordance with the invention should be of values of between −300×10⁻⁶ and 300×10⁻⁶ (values based on the MKS (metre, kilogram, second) system without units). Advantageously susceptibility should be between −100×10⁻⁶ and 100×10⁻⁶, quite preferably between −50×10⁻⁶ and 40×10⁻⁶, still more preferably between −20×10⁻⁶ and 10×10⁻⁶. In particular magnetic field strength (magnetic flux density) of the magnetic resonance tomographs and MR-sequence parameters such as for example excitation angle, echo time, and read-out band width, are to be mentioned as additional influencing factors. The orientation of an endoprosthesis with respect to the main magnetic field of a nuclear spin tomograph also plays a part in regard to the magnitude of a susceptibility artefact which possibly occurs. Set out hereinafter is an example of many possible options for the choice of a metal alloy which satisfies the prerequisites for the manufacture of a fully MR-compatible endoprosthesis (figures given in percent by mass):
Au 20.0-80.0%, alternatively 30.0-60.0%, further alternatively 30-40%,
Cu 20.0-80.0%, alternatively 30.0-60.0%, further alternatively 50-60%,
Pt 0-7.5%, alternatively 1-5%, further alternatively 1-3%,
Pd 0-10%, alternatively 1-7.5%, further alternatively 1-4%,
Ir 0-5%, alternatively 0-4%, further alternatively 0-2%,
Ag 0-20%, alternatively 1-10%, further alternatively 5-10%,
Zn 0-5%, alternatively 0-4%, further alternatively 0-2%,
Sn 0-5%, alternatively 0-4%, further alternatively 0-2%,
Ru 0-5%, alternatively 0-4%, further alternatively 0-2%, further substances a total of less than 15%, preferably below 10%.

The further substances are for example bismuth, antimony, indium, thallium, gold, mercury, beryllium, silver, gallium, tin, carbon, phosphorus, selenium, aluminium, aluminium oxide, silicon, silicon oxide, lead, zinc, sulphur, magnesium oxide, magnesium, zirconium oxide, zirconium, germanium, silicone, rubidium, caesium, magnesium, yttrium, yttrium oxide, tungsten, molybdenum, rhodium, tantalum, titanium, niobium, platinum, vanadium or palladium. The choice of those substances was made on the basis of the susceptibility inherent therein, which is in the appropriate range according to the experiences of the inventors. In this respect it should be expressly pointed out that those substances are not a complete list of all substances considered.

Preferred endoprostheses according to the invention are of the following compositions: for example: 35% Au, 54.4% Cu, 2.2% Pt, 1% Pd, 6.7% Ag, 0.6% Sn, 0.05% Ir,
or: 10% Ag, 90% Cu
or: 50% Ag, 50% Cu
or: 10% Ni, 90% Cu
or: 5% Sn, 95% Cu
or: 60% Pd, 40% Ag.

It will be noted that, in principle, the operating principle is also operative when using pure substances, as tests with Cu and Au have shown. In addition all metals and metal alloys involving a magnetic susceptibility similar to human tissue are suitable as the material for making the endoprosthesis set forth. They are for example: copper, gold, copper-gold alloys and silver-palladium alloys.

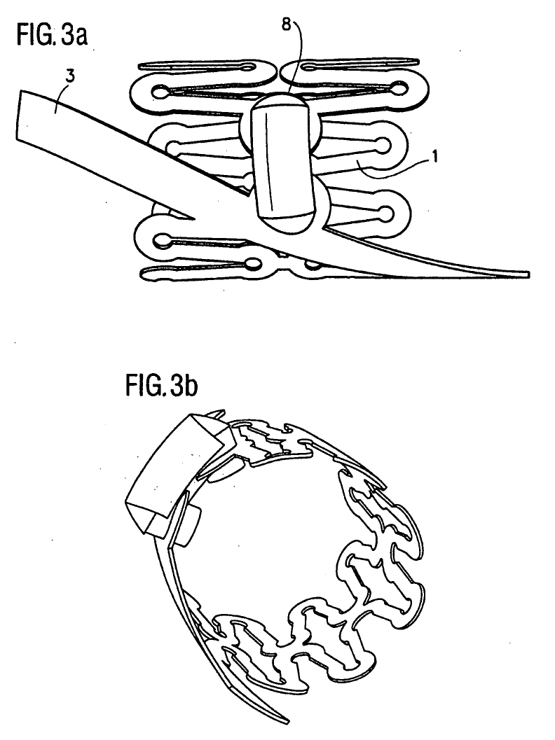

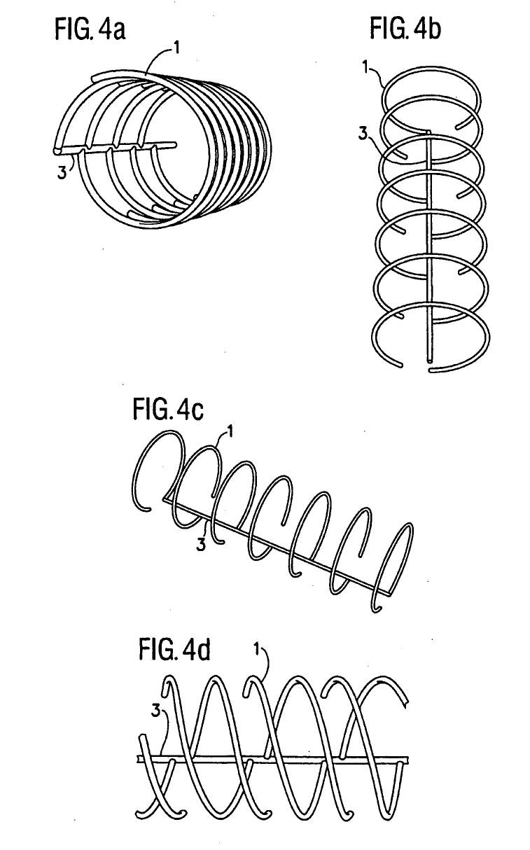

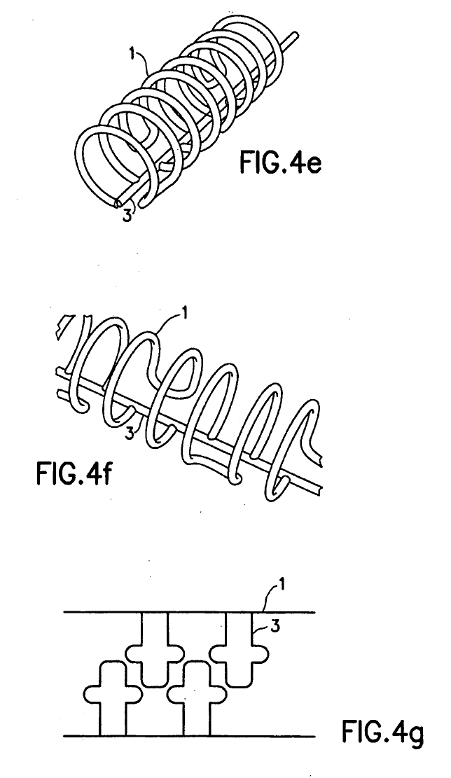

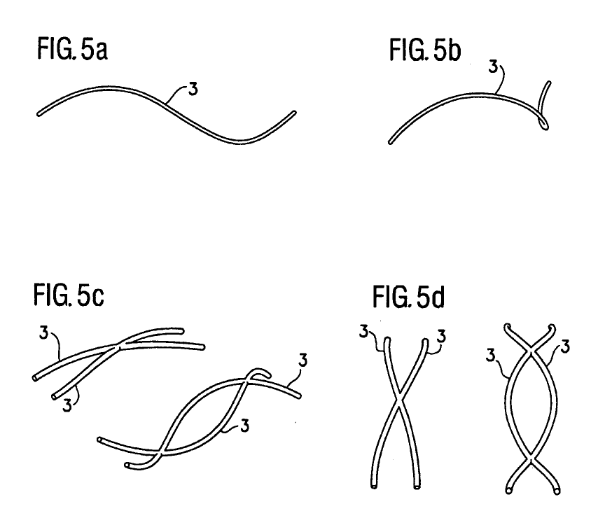

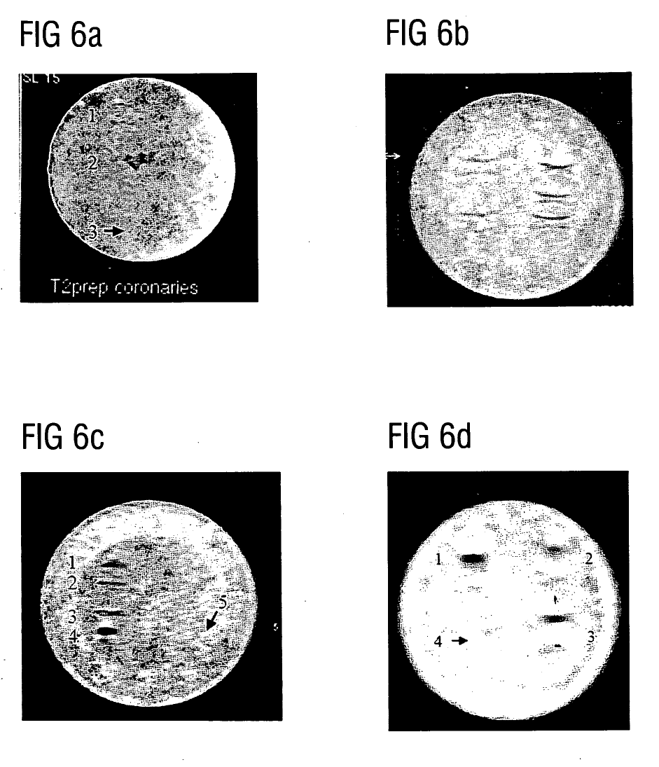

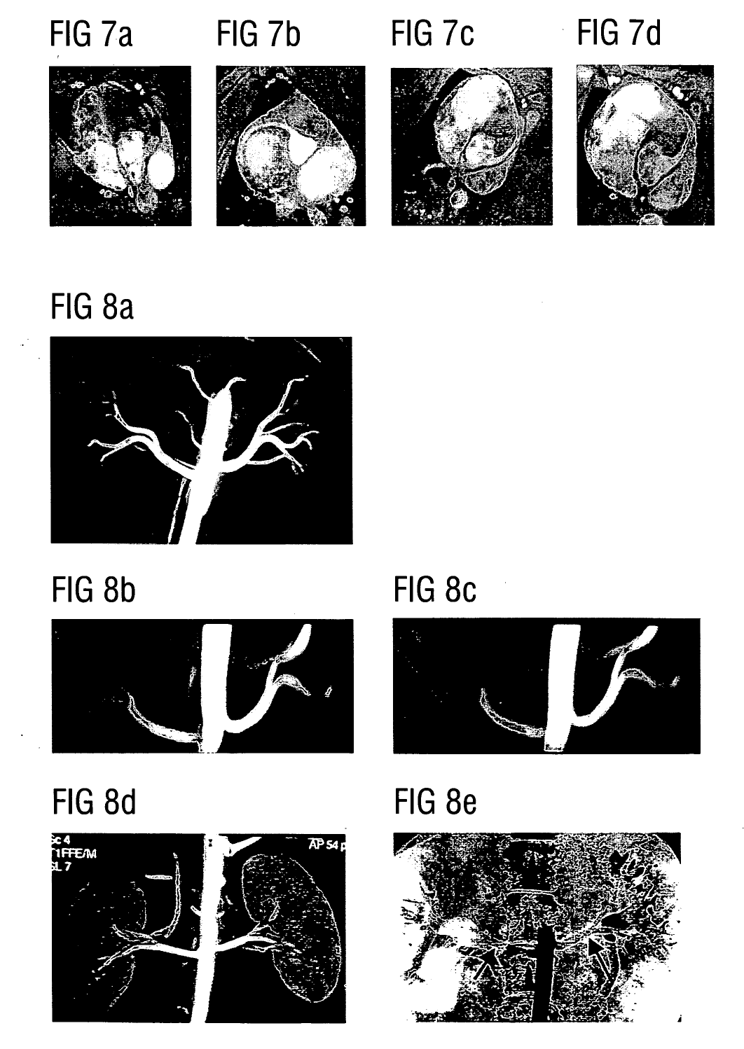

If the endoprosthesis is produced from a tube or flat sheet—which is usually advantageously effected by lasering—the endoprosthesis design should be so selected that, after expansion of the endoprosthesis, as far as possible no circulating current flow can occur, which could shield the interior of the endoprosthesis. In particular the formation of closed circuit structures over the entire circumference of the endoprosthesis by the endoprosthesis bars and a corresponding flow of current in a plane perpendicularly or almost perpendicularly to the longitudinal axis of the endoprosthesis is to be avoided. For that purpose the individual endoprosthesis bars are not brought together in a circular configuration in a plane substantially perpendicularly to the longitudinal axis of the endoprosthesis, but are arranged in displaced relationship or in directly mutually oppositely disposed relationship, without however having a continuous electrical connection with each other. The endoprosthesis bars can be arranged in mutually parallel relationship, perpendicularly or at any angles (FIGS. 2a-e, 4a-d) with any shape for the endoprosthesis bars, starting from the backbone or backbones. The endoprosthesis bars can be in the form of individual bars or in the form of closed or open loop-like or polygonal structures comprising one or more segments (FIGS. 2a-e, 4a-g), without however forming a complete circle around the entire circumference in a plane substantially perpendicularly to the longitudinal axis of the endoprosthesis. In the case of a polygonal structure being formed, the bars can be so shaped that rounded angles are produced. The design can be so selected that those endoprosthesis bars expand perpendicularly and/or parallel to the longitudinal axis of the endoprosthesis. To improve the radial force or to improve uniform expandability, it is possible to provide additional connecting bars which are arranged in any manner between the components of the endoprosthesis and which are of any shape and which are not electrically conducting or which are only very poor conductors (FIG. 3). They can be produced by insulating the bars with for example polytetrafluoroethylene (PTFE), polyethylene, polyamide, polyparaxylylene, polyurethane, and insulating polymers or monomers. They can be joined by welding, glueing, knotting or any other process, to the metallic structure of the endoprosthesis, in each case without full MR-compatibility being limited thereby. A backbone is required along the longitudinal axis of the endoprosthesis, for connecting the individual endoprosthesis bars to each other. The backbone can extend substantially straight (FIG. 4) or curved (FIGS. 1, 2, 3 and 5) or in a polygonal configuration, without MR-compatibility of the endoprosthesis being impaired. The endoprostheses may have either one or more such backbones which are either straight or are of any shape, in which case a helix is advantageous (FIGS. 1-3, 5). If a plurality of backbones are used, they are preferably to be arranged with only one intersection location (FIGS. 5c and d) or with intersection locations which are as far away from each other as possible. Circles or circuits extending perpendicularly to the longitudinal axis of the endoprosthesis are also to be avoided as much as possible when connecting the backbones. Accordingly for example with two sinusoidally extending backbones, the connection between those two backbones should be implemented only at even or odd multiples off 90° (FIGS. 5c, d). Depending on the respectively desired radial force and coverage area by the endoprosthesis, the individual backbones can be provided with the various above-described shapes of endoprosthesis bars.

If the endoprosthesis is not produced from a flat plate or tube but from a wire or a plurality of wires, the wires are provided with a substantially electrical insulation. In principle, it is desirable here to use a biocompatible coating which is electrically non-conducting or slightly current-conducting. Preferably the insulation is at the contact locations and quite preferably involves over 80% of the wire. Preferred materials for the insulation are plastic materials such as polytetrafluoroethylene (PTFE), polyethylene, polyamide, polyparaxylylene, polyurethane, and insulating polymers or monomers. Substantially closed circles or circuits perpendicularly to the longitudinal axis of the endoprosthesis are to be avoided, which is achieved by substantially orienting the wire or wires along the longitudinal axis of the prosthesis. In that respect, to achieve and ensure full MR-compatibility, it is immaterial whether the endoprosthesis is produced by braiding or weaving or from however many individual wires.

The endoprostheses can be singly or multiply coated on the inside and/or outside with one or more substances which can be effective in part or overall as a substance medically or also non-medically and which are either permanently bonded and/or are delivered over time. The coatings can comprise for example fat-soluble vitamins A, D, E and K and derivatives thereof, cortisone and derivatives thereof, heparin and derivatives thereof, immunosuppressives or chemotherapeutic agents. The endoprosthesis can also be provided with a casing with one or more membranes inside and/or outside the endoprosthesis. For example PTFE, polyurethane or polyester are to be mentioned as casing materials. The coatings or casings at the inside and/or outside each have no influence on full MR-compatibility. Modifications of that kind can therefore be effected to improve the general stent properties, without that causing impairment of full MR-compatibility of the endoprostheses. The endoprostheses can also be provided with markers for better visualisation under X-ray radioscopy and/or in magnetic resonance tomography. Examples of such markers are gold rings or rings of lanthanides or very small iron particles.

What is claimed is:

1. An endoprosthesis comprising a woven or braided metallic material having a magnetic susceptibility in the range of between −300×10⁻⁶ and 300×10⁻⁶, wherein the endoprosthesis has an endoprosthesis longitudinal axis and a circumference and is of such a configuration that individual endoprosthesis bars or wires are so oriented along the endoprosthesis longitudinal axis that they form substantially no continuous electrical circuit in a plane which is oriented substantially perpendicularly to the longitudinal axis of the endoprosthesis, over the circumference of the endoprosthesis.

2. An endoprosthesis according to claim 1 where individual endoprosthesis bars extend from one or more backbones.

3. An endoprosthesis according to claim 2 wherein the backbone or backbones are substantially straight.

4. An endoprosthesis according to claim 2 wherein the backbone or backbones are substantially helical.

5. An endoprosthesis according to claim 1, wherein the endoprosthesis bars are formed as individual bars or are in the form of a closed or open polygonal structure.

6. An endoprosthesis according to claim 1 wherein the endoprosthesis has one or more segments.

7. An endoprosthesis according to claim 1 wherein endoprosthesis bars extend substantially perpendicularly or at any angle from the backbone or backbones.

8. An endoprosthesis according to claim 1 wherein the endoprosthesis is produced from one or more substantially insulated wires which are oriented substantially along the longitudinal axis.

9. An endoprosthesis according to claim 8 wherein the wires are electrically insulated at least at the contact locations.

10. An endoprosthesis according to claim 8 wherein at least 80% of the wire or wires is electrically insulated.

11. An endoprosthesis according to claim 1 wherein connecting bars of a substantially insulating material are disposed between the endoprosthesis bars.

12. An endoprosthesis according to claim 1 wherein connecting bars of a slightly current-conducting material are disposed between the endoprosthesis bars.

13. An endoprosthesis according to claim 1 wherein the metallic material includes:
Au 20.0-80.0%
Cu 20.0-80.0%
Pt 0-7.5%
Pd 0-10%
Ir 0-5%
Ag 0-20%
Zn 0-5%
Sn 0-5%
Ru 0-5%
Further substances a total of 0 to less than 15%.

14. An endoprosthesis according to claim 13 wherein the further substances are selected from bismuth, antimony, indium, thallium, gold, mercury, beryllium, silver, gallium, tin, carbon, phosphorus, selenium, aluminium, aluminium oxide, silicon, silicon oxide, lead, zinc, sulphur, magnesium oxide, magnesium, zirconium oxide, zirconium, germanium, silicone, rubidium, caesium, magnesium, yttrium, yttriumoxide, tungsten, molybdenum, rhodium, tantalum, titanium, niobium, platinum, vanadium or palladium.

15. An endoprosthesis according to claim 1 wherein the endoprosthesis is singly or multiply coated.

16. An endoprosthesis according to claim 1 wherein one of the coating contains the fat-soluble vitamins A, D, E and K and derivatives thereof, cortisone and derivatives thereof, heparin and derivatives thereof, immunosuppressives or therapeutic agents.

17. An endoprosthesis according to claim 1 wherein the endoprosthesis is surrounded by a casing comprising one or more membranes.

18. An endoprosthesis formed from a metallic material and having a longitudinal axis and a circumference, the endoprosthesis comprising at least one backbone extending along the longitudinal axis and a plurality of individual endoprosthesis bars or wires extending from the at least one backbone so oriented along the endoprosthesis longitudinal axis that they form substantially no continuous electrical circuit in a plane which is oriented substantially perpendicularly to the longitudinal axis of the endoprosthesis, over the circumference of the endoprosthesis.

19. An endoprosthesis according to claim 18 wherein the at least one backbone is substantially straight.
